(19) United States
(12) Patent Application Publication
(10) Pub. No.: US 2024/0081720 A1
(43) Pub. Date: Mar. 14, 2024

(54) METHOD AND APPARATUS FOR DIAGNOSING DIZZINESS THROUGH EYE MOVEMENT MEASUREMENT BASED ON VIRTUAL REALITY, RECORDING MEDIUM STORING PROGRAM FOR REALIZING THE SAME, AND COMPUTER PROGRAM STORED IN RECORDING MEDIUM

(71) Applicants: Industry Academic Cooperation Foundation, Hallym University, Chuncheon-Si (KR); NEUROEARS CO., LTD., Incheon (KR)

(72) Inventors: Sung Kwang Hong, Seongnam-si (KR); Ha Neul Kwon, Incheon (KR)

(73) Assignees: Industry Academic Cooperation Foundation, Hallym University, Chuncheon-Si (KR); NEUROEARS CO., LTD., Incheon (KR)

(21) Appl. No.: 18/261,386

(22) PCT Filed: Jun. 8, 2022
(86) PCT No.: PCT/KR2022/008044

§ 371 Date: Jul. 13, 2023

(30) Foreign Application Priority Data
Dec. 22, 2021 (KR) 10-2021-0185393

(51) Int. Cl. A61B 5/00; A61B 5/11

(52) U.S. Cl. CPC A61B 5/4023; A61B 5/1103; A61B 5/1116; A61B 5/4863; A61B 5/7203; A61B 5/742

(57) ABSTRACT

The present invention relates to a technology for diagnosing dizziness by testing eye movement (motion), and more particularly, to a method and apparatus for diagnosing dizziness through eye movement measurement based on virtual reality capable of utilizing a commercialized virtual reality device to measure and test eye movement and transmit resultant data, measuring optokinetic nystagmus through movement of various gazing points (objects) generated in virtual reality, and diagnosing dizziness through the measurement, a recording medium storing a program for realizing the same, and a computer program stored in the recording medium.

### CROSS-REFERENCE TO RELATED APPLICATION

This application claims priority to and the benefit of Korean Patent Application No. 10-2021-0185393, filed on Dec. 22, 2021, the disclosure of which is incorporated herein by reference in its entirety.

### BACKGROUND

### 1. Field of the Invention

The present invention relates to a technology for diagnosing dizziness by testing eye movement (motion) and, more particularly, to a method and apparatus for diagnosing dizziness through eye movement measurement based on virtual reality capable of utilizing a commercialized virtual reality device to measure and test eye movement and transmit resultant data, measuring optokinetic nystagmus through the movement of various gazing points (objects) generated in virtual reality, and diagnosing dizziness through the measurement, a recording medium storing a program for realizing the same, and a computer program stored in the recording medium.

### 2. Discussion of Related Art

Dizziness refers to all the symptoms of feeling as if a person or surrounding objects are moving even if they are still. It is mainly caused by abnormalities in the peripheral vestibular system or a central vestibular system. Clinically, the dizziness is determined based on whether a vestibular function is abnormal through an interaction between the vestibular system and vision, that is, the vestibular-ocular reflex. The vestibular-ocular reflex refers to the ability to fix gaze regardless of a change in a head position, and when there is the abnormality in the vestibular system, people suffer from dizziness as if the world is moving due to a loss of visual fixation. Although the vestibular-ocular reflex has the ability to fix gaze in response to the rapid change in the head position, but it has been known that the vestibular-ocular reflex does not play a large role in the extremely slow change (rotation) in the head position. Accordingly, an oculomotor function is responsible for visual fixation of the extremely slow change in the head position that the vestibular-ocular reflex is not responsible for. Accordingly, a human body recognizes space and maintains balance through maintaining proper mutual functions of the oculomotor function and the vestibular-ocular reflex.

A video nystagmographynystagmography is commonly used as an apparatus for testing whether there are functional abnormalities in the vestibular-ocular reflex and the oculomotor function. However, the nystagmography is manufactured based on hardware, and therefore, manufactured at a relatively high price. Due to the economic burden caused by the expensive nystagmography, many restrictions are inevitably followed. Therefore, an alternative technology is needed to replace the expensive nystagmography and test whether there are the functional abnormalities in the vestibular-ocular reflex and the oculomotor function.

As an alternative technology to the existing nystagmography, a currently commercialized headset-type virtual reality device may be used. Most virtual reality devices are provided with a function of tracking eye movement. Most of these functions of tracking eye movement are used for game purposes or marketing purposes, and are also partially used for medical purposes with limitation. In addition, since existing medical devices for measuring nystagmus have structures substantially similar to currently commercialized head mounted display (HMD), the existing medical devices can be used as the nystagmography. However, eye trackers attached to most virtual reality devices can track only a vector, and cannot practically transmit eye images to the outside based thereon.

### RELATED ART DOCUMENT

[Patent Document]
(Patent Document 1) KR 10-1978548 B1. May 8. 2019
(Patent Document 2) KR 10-1898414 B1, Sep. 6, 2018

### SUMMARY OF THE INVENTION

An object of the present invention provides an apparatus and method for diagnosing dizziness capable of measuring and testing vestibular-ocular reflex and optokinetic nystagmus by replacing an expensive nystagmography used to test whether there are functional abnormalities in vestibular-ocular reflex and an oculomotor function.

Another object of the present invention provides an apparatus and method for diagnosing dizziness capable of testing vestibular-ocular reflex and optokinetic nystagmus using a virtual reality device.

Another object of the present invention provides an apparatus and method for diagnosing dizziness capable of measuring vestibular-ocular reflex by detecting a position and movement of a head, and testing optokinetic nystagmus by processing raw data acquired through an eye tracking function and an internal camera.

Another object of the present invention provides a recording medium storing a program for realizing the method of diagnosing dizziness is stored and a computer program stored in the recording medium.

In addition, the present invention is not limited to the above-described purpose, and various objects may be additionally provided through technologies described through embodiments and claims to be described later.

In an aspect of the present invention, an apparatus for diagnosing dizziness through eye movement measurement based on virtual reality includes a head movement detection unit configured to receive a signal detected from a head tracker attached to a virtual reality device and detecting a head position and movement of a patient to detect head movement of the patient, an eye movement video acquisition unit configured to receive raw data obtained by capturing eye movement of the patient from an eye tracker attached to the virtual reality device to acquire eye movement video, an eye movement video transmitting unit configured to transmit the eye movement video acquired by the eye movement video acquisition unit, a nystagmus detection unit configured to receive the eye movement video to filter saccadic oscillation in the eye movement video to detect nystagmus and filter meaningless eye movement that is not measured since an eye moves too slow or is covered due to blinking in the detected nystagmus to detect only meaningful nystagmus, and a diagnostic unit configured to diagnose the dizziness of the patient based on the meaningful nystagmus detected by the nystagmus detection unit.

The eye movement video acquisition unit may acquire the eye movement video by processing the raw data of the eye movement captured using an internal camera of the eye tracker using a processing program.

The apparatus may further include an arithmetic unit configured to calculate a relative ratio between the head movement and the eye movement by dividing a movement angle of a head detected by the head movement detection unit in response to a change in the patient's head by the eye movement angle obtained in response to the change in the head.

The diagnostic unit may determine that there is an abnormality in a vestibular function when the relative ratio calculated by the arithmetic unit is not “.”

The apparatus may further include a graph output unit configured to display the nystagmus detected by the nystagmus detection unit in three axes (horizon/vertical/torsional axes).

The apparatus may further include a gaze guidance gazing point providing unit configured to provide a gazing point for gaze guidance through a display installed in the virtual reality device.

The gazing point may have a curtain shape or a point shape.

The apparatus may further include a posture adjustment guide unit configured to provide the patient with an accurate posture required for each test through the virtual reality device.

In another aspect of the present invention, a method of diagnosing dizziness through eye movement measurement based on virtual reality includes (a) receiving a signal detected from a head tracker attached to a virtual reality device and detecting a head position and movement of a patient to detect the head movement of the patient, (b) receiving raw data obtained by capturing the eye movement of the patient from an eye tracker attached to the virtual reality device to acquire and transmit eye movement video, (c) distinguishing saccadic oscillation and nystagmus in the eye movement video and filtering meaningless eye movement that is not measured since an eye moves too slow or is covered due to blinking in the detected nystagmus to detect only meaningful nystagmus, and (d) diagnosing a type of dizziness of the patient based on the detected meaningful nystagmus.

In the operation (b), the eye movement video may be acquired and transmitted by processing the raw data of the eye movement captured using an internal camera of the eye tracker using a processing program.

In the operation (d), a relative ratio between the head movement and the eye movement may be calculated by dividing a movement angle of a head detected in response to the change in the head in the operation (a) by the eye movement angle moving in response to the change in the head, and it may be determined that there is abnormality in a vestibular function when the calculated relative ratio does not reach “I” or a normal value that is predetermined.

The method may further include (e) displaying the nystagmus detected in the operation (c) in three axes (horizon/vertical/torsion axes).

The method may further include, before the operation (b), (f) providing a gazing point for gaze guidance through the virtual reality device.

The gazing point may have a curtain shape or a point shape.

The method may further include, after the operation (a), (g) providing the patient with an accurate posture required for each test through the virtual reality device.

In addition, there is provided a computer-readable recording medium in which a program for realizing the method of diagnosing dizziness through eye movement measurement based on virtual reality according to another embodiment of the present invention in order to achieve the above object is stored.

In addition, there is provided a computer program stored in a computer-readable recording medium storing a program for realizing the method of diagnosing dizziness through eye movement measurement based on virtual reality according to another embodiment of the present invention in order to achieve the above object.

### BRIEF DESCRIPTION OF THE DRAWINGS

FIG. 1 is a schematic block diagram illustrating an apparatus for diagnosing dizziness according to an embodiment of the present invention.

FIG. 2 is a schematic diagram illustrating an example of a configuration of a virtual reality device illustrated in FIG. 1.

FIG. 3 is a schematic diagram illustrating a hardware configuration of an eye tracker illustrated in FIG. 1.

FIG. 4 is a schematic diagram illustrating a raw signal data processing process according to the present invention.

FIGS. 5 and 6 are diagrams illustrating nystagmus graphs according to the present invention.

FIGS. 7A and 7B are schematic diagrams illustrating a gazing point according to the present invention.

FIG. 8 is a flowchart illustrating a method of diagnosing dizziness according to an embodiment of the present invention.

FIG. 9 is a flowchart illustrating an additional process of a method of diagnosing dizziness according to an embodiment of the present invention.

FIG. 10 is a flowchart illustrating an additional process of a method of diagnosing dizziness according to an embodiment of the present invention.

### DETAILED DESCRIPTION OF EXEMPLARY EMBODIMENTS

Various advantages and features of the present invention and methods accomplishing them will become apparent from the following description of embodiments with reference to the accompanying drawings. However, the present invention is not limited to embodiments disclosed below, but is embodied in a variety of different forms, and is provided to fully inform those skilled in the art of the scope of the invention to which the present invention belongs, and the present invention is defined by the scope of the claims.

In addition, terms used in the present specification are for explaining exemplary embodiments rather than limiting the present invention. Unless otherwise stated, a singular form includes a plural form in the present specification. For example, the terms “comprises” (or “includes”) and/or “comprising” (or “including”) used in the specification may add the mentioned components and processes. Throughout the specification, like reference numerals denote like elements. The term “and/or”includes each and every combination of one or more of the mentioned items.

Unless defined otherwise, all terms (including technical and scientific terms) used in the present specification have the same meaning as meanings commonly understood by those skilled in the art to which the present invention pertains. In addition, terms defined in commonly used dictionary are not ideally or excessively interpreted unless explicitly defined otherwise.

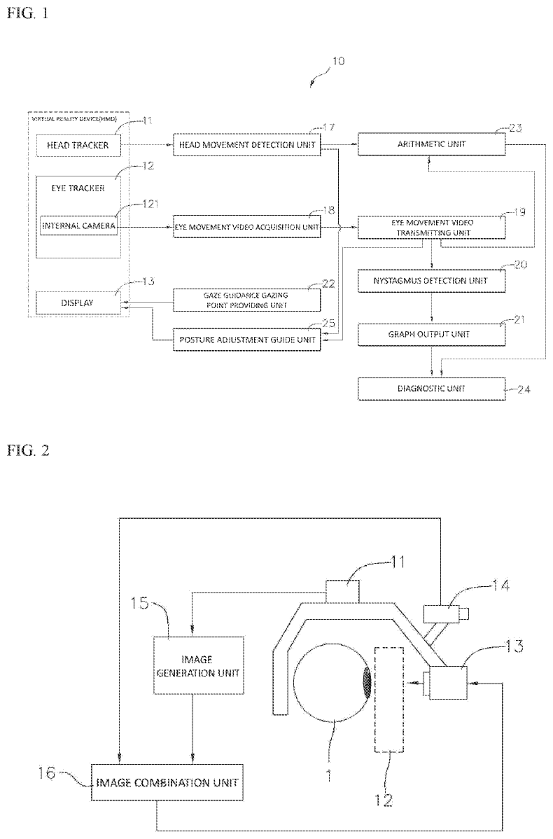

FIG. 1 is a schematic block diagram illustrating an apparatus for diagnosing dizziness according to an embodiment of the present invention.

Referring to FIG. 1, an apparatus 10 for diagnosing dizziness according to an embodiment of the present invention measures a position and movement of a head using a commercialized virtual reality device, for example, a head mounted display (HMD), and measures eye movement (motion) and transmits the measured eye movement to the outside to test vestibular-ocular reflex and optokinetic nystagmus. In this way, it is possible to replace a nystagmography, which is widely used as an existing eye movement test device, by using a commercialized virtual reality device.

FIG. 2 is a schematic diagram illustrating an example of a configuration of a virtual reality device illustrated in FIG. 1.

As illustrated in FIGS. 1 and 2, a virtual reality device according to the present invention includes a head tracker 11 that tracks a position and movement of a head, an eye tracker 12 that tracks eye movement, and a display (monitor) 13 that outputs an image to a patient, and the virtual reality device may further include a video camera 14 that captures a real world, an image generation unit 15 that generates images (e.g., graphic images) corresponding to the position and movement of the head tracked by the head tracker 11, and an image combination unit 16 that combines an image of the real world captured by the video camera 14 and an image generated by the image generation unit 15 and transmits the combined image to the display 13.

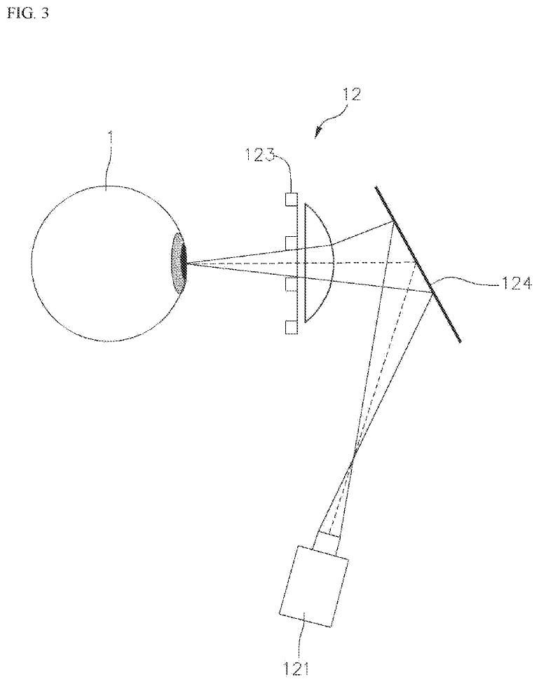

FIG. 3 is a schematic diagram illustrating a hardware configuration of an eye tracker illustrated in FIG. 1.

Referring to FIG. 3, the eye tracker 12 includes an internal camera 121 that captures movement of an eye 1. In this case, the internal camera 121 may capture the movement of the eye 1 through a reflection mirror 124, and as a light source 123, for example, a near infrared (NIR) light source may be used.

As illustrated in FIG. 1, the head tracker 11 includes a sensor that detects the position and movement of the head, and the position and movement of the head tracked through the sensor are detected through the head movement detection unit 17. That is, the head movement detection unit 17 detects the position and movement of the head tracked by the head tracker 11 and processes the position and movement information of the head.

Referring to FIG. 1, as an example in the embodiment of the present invention, the head movement detection unit 17 is configured separately from the head tracker 11, and may be integrated into the head tracker 11. That is, the head tracker 11 is composed of a sensor that detects the position and movement of the head, and the head movement detection unit 17 is software that receives a detection signal detected through the sensor to detect the position and movement of the head.

In addition, the apparatus 10 for diagnosing dizziness according to the embodiment of the present invention includes an eye movement video acquisition unit 18 for processing the eye movement tracked by the eye tracker 12.

The eye movement video acquisition unit 18 may be integrated into the eye tracker 12 and operated, and recombine the raw signal data of the eye 1 captured through the internal camera 121 to transmit the eye movement video, that is, the eye movement video to the outside.

An example of the form of the raw signal data transmitted from the internal camera 121 is as follows.

TABLE 1
Structure (numerical value (data) of bool,
int, float, vector2, and vector3)

Presence/absence of user (bool)
Timestamp[ms] (int)
Gaze start position (vector3)
Normalized gaze direction [0-1] (vector3)
Diameter of pupil (mm)(float)
Degree of eye opening [0-1] (float)
Gaze position on screen [0-1] (vector2)

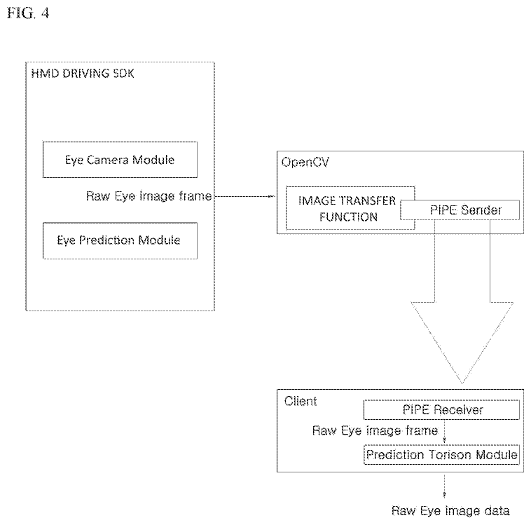

FIG. 4 is a schematic diagram illustrating a raw signal data processing process according to the present invention.

Referring to FIGS. 1 and 4, when an eye moves, a process of confirming the movement of the eye (pupil) by tracking the vertical, horizontal, and torsional aspects of the eye based on the raw signal data provided from the internal camera 121 is required. However, a software development kit (SDK) provided by general HMD manufacturers do not provide eye rotation (torsional) information. In order to obtain the torsional information of the eye, the image of the eye movement inside needs to be transmitted to the outside in real time while wearing the HMD, and the torsional information needs to be confirmed through self-image processing.

As illustrated in FIG. 4, a processing program is required to process raw data captured through the internal camera 121 of the eye tracker 12. For example, the SDK of the HMD may be used as the processing program. In addition, the SDK processing program uses an eye camera module and an eye prediction module in the form of software to receive and process the eye image information captured through the internal camera 121 for eye tracking, which are a common structure.

The SDK generates an image transfer function using openCV, divides the eye image captured through the internal camera 121 into image frames, and transmits the divided image frames to the eye prediction module. In this case, the image frame is modified to load image frame data into a PIPE by modifying the openCV function and transmit the image frame data. Then, a receiving end of the PIPE may be implemented at a client side to extract an image frame (raw eye image data).

After receiving signals transmitted from the eye tracker 12, the eye movement video acquisition unit 18 recombines the signals in the same processing method as illustrated in FIG. 4 to acquire an actual eye movement image, and transmits the acquired eye movement image through the eye movement video transmitting unit 19. Meanwhile, as illustrated in FIG. 1, the nystagmus detection unit 20 is for detecting the oculomotor function, and detects meaningful nystagmus, which is required to diagnose dizziness, from the eye movement in the eye movement video provided through the eye movement video transmitting unit 19.

The nystagmus refers to involuntary movement of the eye. For example, when sitting on a chair that rotates to the right, eyes repeat the movement of turning to the right and then returning to an original state according to a rotating speed of the chair, although we are not aware of it. In this case, the eye movement that usually returns to its original state returns quickly, which is called “fast component.” Thus, the nystagmus is composed of “fast component” and “slow component.” However, although it looks like the nystagmus, there is an eye movement that is not the nystagmus, such as the eye moving quickly to the right and then quickly returning to its original state, or moving slowly to the right and then slowly returning to the left. This is not called the nystagmus but is usually called “saccadic oscillation.” Since the saccadic oscillation is a pathologic finding suggesting abnormality in the central-vestibular-optic pathway, it is very important to distinguish the saccadic oscillation from the nystagmus. Also, even with the nystagmus, when intensity (speed of the eye movement of the slow component) is too weak below 2-3 deg/sec or when an eye is covered due to blinking or the like, the nystagmus may not be properly measured, and the same goes for the saccadic oscillation.

Therefore, the nystagmus detection unit 20 filters the eye movement of the eye movement video transmitted through the eye movement video transmitting unit 19 to detect only the meaningful nystagmus. That is, the nystagmus detection unit 20 may distinguish between the saccadic oscillation and the nystagmus, and includes a function of detecting only nystagmus excluding the meaningless eye movement (movement that is not measured since eyes are blocked due to too slow, blinking, or the like).

In addition, since the nystagmus is the eye movement along a time axis, when the nystagmus is not detected for a specific period of time, such as when a patient closes his or her eyes, the nystagmus also includes a function of performing prediction through a pattern immediately before closing the eyes and after opening the eyes and performing measurement again. Describing an example of the case of navigation used while driving a car, even if GPS does not operate after entering the tunnel, the movement pattern of the nystagmus may be predicted by the same method as the method of displaying a car's route at a speed and location before entering the tunnel. In addition, in the case of the saccadic oscillation, it may include a function of warning to stop the test and recommending videography.

The meaningful nystagmus detected through the nystagmus detection unit 20 is transmitted to the graph output unit 21.

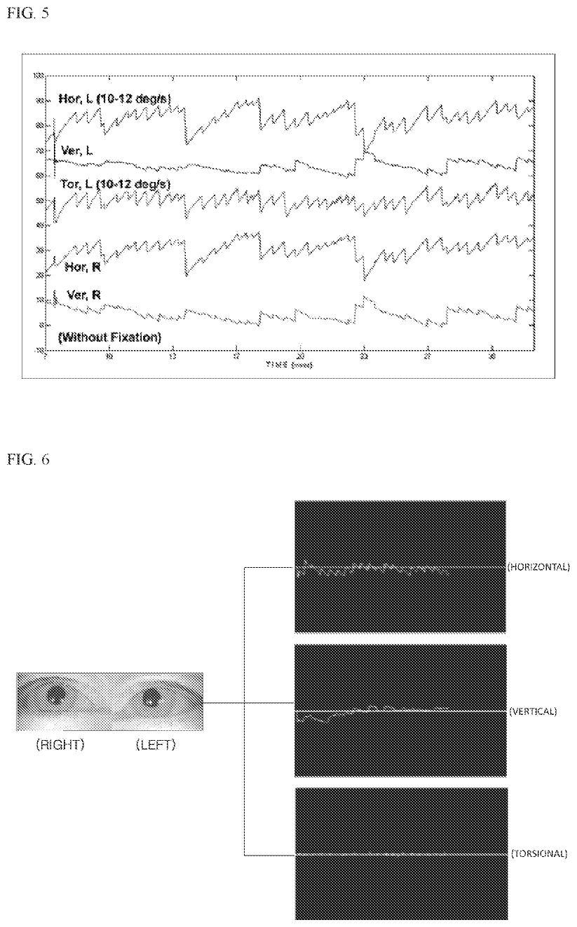

FIGS. 5 and 6 are diagrams illustrating nystagmus graphs according to the present invention.

As illustrated in FIGS. 1 and 5, the graph output unit 21 generates and outputs a nystagmus graph for the patient based on the nystagmus detected through the nystagmus detection unit 20. The nystagmus graph is a graph of eye movement over time, and the eye moves along three axes: horizontal (right/left), vertical (up/down), and torsional (clockwise/counterclockwise). Here, in FIG. 5, horizontal (Hor) left (L) means a left eye's horizontal movement, vertical (Ver) left (L) means the left eye's vertical movement, and torsional (Tor) left (L) means the left eye's torsion. The same goes for a right eye. In addition, as illustrated in FIG. 6, the eye graph is divided into three movements such as horizontal movement, vertical movement, and torsional and displayed.

Meanwhile, as illustrated in FIG. 1, the apparatus 10 for diagnosing dizziness according to the embodiment of the present invention may further include a gaze guidance gazing point providing unit 22. The gaze guidance gazing point providing unit 22 provides a gazing point (target point) for gaze guidance in virtual reality to measure the optokinetic nystagmus.

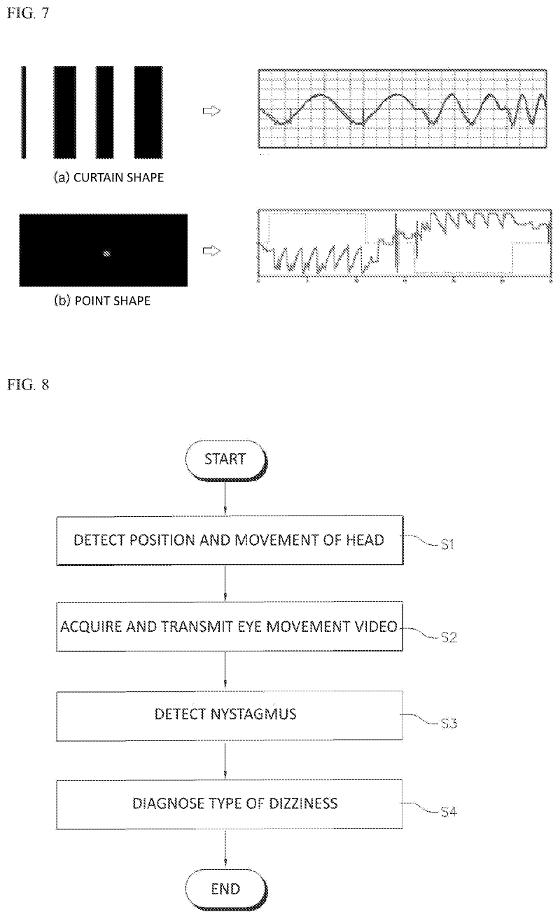

FIGS. 7A and 7B are schematic diagrams illustrating an example of a gazing point according to the present invention.

As illustrated in FIGS. 1 and 7, the gaze guidance gazing point providing unit 22 may output the gazing point for gaze guidance in virtual reality through the display 13 in order to measure the optokinetic nystagmus. Of course, it may be provided as a separate display device instead of the display 13.

The gaze guidance gazing point providing unit 22 outputs a gazing point having a certain shape through the display 13 to evoke the optokinetic nystagmus, and the gazing point has a curtain shape (see FIG. 7A) or a point shape (see FIG. 7B). In this case, the gazing point may rotate horizontally or vertically at a constant or various speeds, or may be provided in the form of saccades. Then, the movement of the nystagmus moving in response to the gazing point is tracked, and the nystagmus movement tracked in this way may be output to the graph output unit 21.

As illustrated in FIG. 1, the apparatus 10 for diagnosing dizziness according to an embodiment of the present invention may further include an anthmetic unit 23. The arithmetic unit 23 divides the movement angle of the head detected by the head movement detection unit 17 by the eye movement angle to calculate a relative ratio between the head movement and the eye movement. Here, the movement angle of the head is the movement angle of the head that is changed by moving the head of the patient, and refers to the angle at which the head moves based on the reference value (reference position of the initial head). The eye movement angle is an angle of an eye moved corresponding to an angle at which the head of the patient moves due to the change in the patient's head, and may be provided through the eye movement video transmitting unit 19 or the eye movement video acquisition unit 18.

The arithmetic unit 23 calculates a relative ratio obtained by dividing the movement angle of the head detected by the head movement detection unit 17, that is, the movement angle of the head from the reference value by the eye movement angle. Then, the relative ratio calculated by the arithmetic unit 23 is provided to a diagnostic unit 24.

The diagnostic unit 24 determines whether there is the abnormality in the vestibular function using the relative ratio calculated by the arithmetic unit 23. For example, when the head movement detection unit 17 may determine that there is the abnormality in the vestibular function when the value obtained by dividing the movement angle of the head detected in response to the change in the head by the eye movement angle, that is, the calculated relative ratio does not become “1.” For example, when the head moves 20° to the right for 1 second, the eyes normally move 200 for the same time in the opposite direction. For example, when it is assumed that, when the head moves 30° to the right for 1 second, eyes move only 20° during the same time, the arithmetic unit 23 divides the movement angle of the head (20°) by the nystagmus movement angle (30°), and when the divided value does not become “1,” the diagnostic unit 24 determines that there is the abnormality in the vestibular function of the right side. However, this process is a simple example, and the normal value of the calculated relative ratio is not necessarily “1,” which may be determined by experts using it by inputting normal values differently and classifying abnormalities according to the input normal values.

The diagnostic unit 24 may diagnose the type of dizziness of the patient based on the nystagmus detected by the nystagmus detection unit 20. In order to diagnose the type of dizziness of a patient, the information on the dizziness is stored and registered in a database, and the diagnostic unit 24 may compare the currently measured patient's nystagmus with pre-registered dizziness information to diagnose the type of dizziness (symptom).

Meanwhile, the apparatus 10 for diagnosing dizziness according to the embodiment of the present invention further includes a posture adjustment guide unit that provides a test method for an operation of each test and guides a guide in a posture required for an accurate test. The posture adjustment guide unit 25 provides (feedback) a posture required for accurate test to a patient through a speaker (not illustrated).

The feedback provided by the posture adjustment guide unit 25 is shown in Table 2 below.

TABLE 2

| Division | Test Item | Description | Feedback |
|---|---|---|---|
| [1] | Spontaneous | Wear HMD | Alarm |
| | nystagmus | while sitting for certain | when moving |
| | | time and keep that state | during the test |
| | | for certain time until end | |
| | | signal comes out | |
| [2] | Left and right | Look at gazing | Alarm |
| | gaze-evoked | point (eye target point) | when not looking |
| | nystagmus | that is shown in front in | at |
| | | virtual reality | |
| [3] | Head-shaking | Shake head | Alarm |
| | nystabmus | from side to side for | when not as |
| | | about 20 seconds at rate | directed |
| | | of 2-3 times per second | |
| | | and keep that state for | |
| | | certain amount of time | |
| | | until end signal comes | |
| | | out | |
| [4] | Head bowing | Bend neck 90° | alarm |
| | test | while sitting and keep | when neck is |
| | | that state for certain time | lowered less or |
| | | until end signal comes | more |
| | | out | |
| [5] | Lie-down test | Do not move | Alarm |
| | | While lying down until | when not as |
| | | end signal comes out | directed |
| [6] | Test of turning | Turn head to | Alarm |
| | head to right while | right while lying down | when not as |
| | lying down | and keep that state for | directed |
| | | certain time until end | |
| | | signal comes out | |
| [7] | Test of turning | Turn head to left | Alarm |
| | head to left while lying | while lying down and | when not as |
| | down | keep that state for certain | directed |
| | | time until end signal | |
| | | comes out | |
| [8] | Test of turning | Turn entire body | Alarm |
| | entire body and head | to right while lying | when not as |
| | to right while lying | down and keep that state | directed |
| | down | for certain time until end | |
| | | signal comes out | |
| [9] | Test of turning | Turn entire body | Alarm |
| | entire body and head | to left while lying down | when not as |
| | to left while lying | and keep that state for | directed |
| | down | certain time until end | |
| | | signal comes out | |
| [10] | Test of | Keep head | Alarm |
| | keeping head below | below horizontal plane | when not as |
| | horizontal plane while | and keep that state for | directed |
| | lying down | certain time until end | |
| | | signal comes out | |
| [11] | Right Dix- | Turn head 45° to | Alarm |
| | Hallpike maneuver | right and lie still, but | when not as |
| | test | keep head below the | directed |
| | | horizontal plane for | |
| | | certain time until end | |
| | | signal comes out | |
| [12] | Left Dix- | Turn head 45° to | Alarm |
| | Hallpike maneuver | left and lie still, but keep | when not as |
| | test | head below the | directed |
| | | horizontal plane for | |
| | | certain time until end | |
| | | signal comes out | |
| [13] | Smooth | Keep an eye on | Alarm |
| | pursuit eye movement | moving gazing point in | when not as |
| | test | virtual reality | directed |
| [14] | Saccade test | Keep an eye on | Alarm |
| | | moving gazing point in | when not as |
| | | virtual reality | directed |
| [15] | Optokinetic | Keep an eye on | Alarm |
| | Nystagmus | moving gazing point in | when not as |
| | | virtual reality | directed |

FIG. 8 is a flowchart illustrating a method of diagnosing dizziness according to an embodiment of the present invention.

Referring to FIGS. 1 and 8, the head movement is detected using a virtual reality device such as an HMD (S1). That is, the position and movement of the head may be detected using the head tracker 11 attached to the virtual reality device while the HMD having a headset structure is worn on the head.

Subsequently, a video for the eye movement is obtained and transmitted (S2). For example, after the raw data is acquired by capturing the eye of the patient through the eye tracker 12 attached to the virtual reality device, the acquired raw data is acquired using, for example, the processing program as illustrated in FIG. 4 to acquire the eye movement video and transmit the acquired video.

Subsequently, the meaningful nystagmus is detected from an eye of a patient with dizziness (S3). That is, the meaningful nystagmus is detected from the acquired eye movement video. For example, in the acquired eye movement video, the saccadic oscillation is filtered and only the nystagmus is detected, and the meaningless eye movement is filtered in the detected nystagmus to detect only actually meaningful nystagmus.

Subsequently, the type of dizziness of the patient is diagnosed based on the detected meaningful nystagmus (S4). For example, in order to diagnose the type of dizziness of a patient, the information on the dizziness is stored and registered in the database, and the diagnostic unit 24 may compare the currently measured patient's nystagmus with pre-registered dizziness information to diagnose the type of dizziness (symptom).

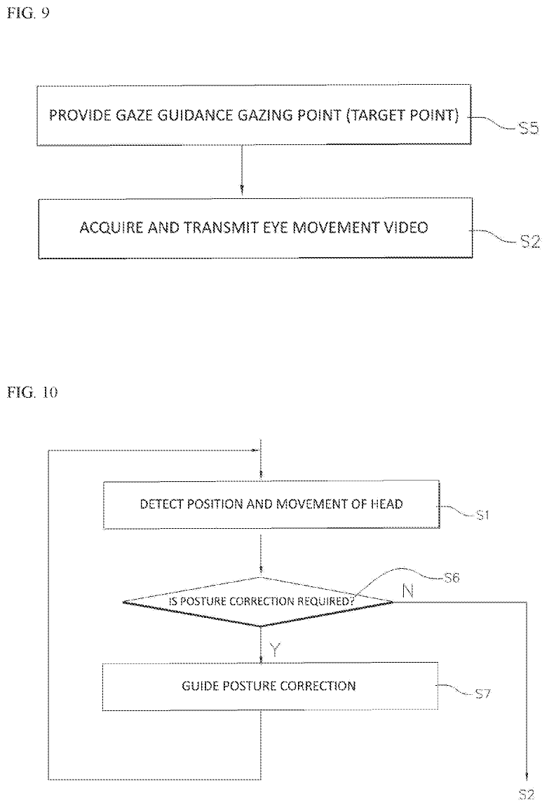

FIG. 9 is a flowchart illustrating an additional process of a method of diagnosing dizziness according to an embodiment of the present invention.

As illustrated in FIG. 9, the method of diagnosing dizziness according to the embodiment of the present invention further includes a process of providing a gaze guidance gazing point (S5) before the process of acquiring and transmitting an eye movement video (S2).

As illustrated in FIGS. 7A and 7B, in the process of providing the gaze guidance gazing point (S5), a gazing point in a curtain shape (see FIG. 7A) having a vertical bar shape or a point shape (see FIG. 7B) is provided.

FIG. 10 is a flowchart illustrating an additional process of a method of diagnosing dizziness according to an embodiment of the present invention.

Referring to FIG. 10, the method of diagnosing dizziness according to the embodiment of the present invention further includes posture correction processes (S6 and S7) of correcting the patient's posture after the process of detecting the head position and movement (S1). For example, the posture correction processes S6 and S7 provide a test method for each operation of each test, and guides a patient in a posture (see Table 2) required to perform an accurate test.

The method of diagnosing dizziness according to the embodiment of the present invention described above may be implemented in the form of a recording medium (or computer program product) executable by a computer, such as a program module stored in a computer-readable medium and executed by a computer, for example.

Here, the computer-readable medium may include a computer storage medium (e.g., a memory, a hard disk, a magnetic/optical medium, a solid-state drive (SSD), or the like). A computer-readable medium may be any available media that may be accessed by a computer and includes both volatile and nonvolatile media and removable and non-removable media.

In addition, the method of diagnosing dizziness according to the embodiment of the present invention includes instructions executable by a computer in whole or in part, and the computer program includes programmable machine instructions processed by a processor, and may be implemented in a high-level programming language, object-oriented programming language, assembly language, or machine language, and the like.

As described above, according to an apparatus and method for diagnosing dizziness through virtual reality-based eye movement measurement according to embodiments of the present invention, by acquiring and transmitting eye movement videos using commercialized virtual reality devices, it is possible to simply test dizziness using a relatively inexpensive virtual reality device. In addition, it is possible to acquire information on a diagnosis of dizziness in an easy way by diagnosing dizziness through a simple test.

In addition, according to an apparatus and method for diagnosing dizziness through virtual reality-based eye movement measurement according to embodiments of the present invention, by recombining raw signal data (raw signal data for vector tracking) captured through an internal camera of an eye tracker to transmit eye movement video to the outside in real time and measure optokinetic nystagmus, it is possible to replace the role of the existing expensive nystagmography, minimize wasted medical costs due to unnecessary imaging tests in case of dizziness patients, and contribute to public health.

Hereinabove, although exemplary embodiments of the present invention have been described and illustrated using specific terms, such terms are only intended to clarify the present invention. It is obvious that various modifications and changes may be made to the embodiments of the present invention and the described terms without departing from the technical spirit and scope of the following claims. Such modified embodiments should not be individually understood from the spirit and scope of the present invention, and it is to be understood that the embodiments of the present invention fall within the scope of the claims of the present invention.

1. An apparatus for diagnosing dizziness through eye movement measurement based on virtual reality, the apparatus comprising:
a head movement detection unit configured to receive a signal detected from a head tracker attached to a virtual reality device and detecting a head position and movement of a patient to detect head movement of the patient;
an eye movement video acquisition unit configured to receive raw data obtained by capturing eye movement of the patient from an eye tracker attached to the virtual reality device to acquire eye movement video;
an eye movement video transmitting unit configured to transmit the eye movement video acquired by the eye movement video acquisition unit;
a nystagmus detection unit configured to receive the eye movement video to filter saccadic oscillation in the eye movement video to detect nystagmus and filter meaningless eye movement that is not measured since an eye moves too slow or is covered due to blinking in the detected nystagmus to detect only meaningful nystagmus; and
a diagnostic unit configured to diagnose a dizziness of the patient based on the meaningful nystagmus detected by the nystagmus detection unit.

2. The apparatus of claim 1, wherein the eye movement video acquisition unit acquires the eye movement video by processing the raw data of the eye movement captured using an internal camera of the eye tracker using a processing program.

3. The apparatus of claim 1, further comprising an arithmetic unit configured to calculate a relative ratio between the head movement and the eye movement by dividing a movement angle of a head detected by the head movement detection unit in response to a change in the patient's head by the eye movement angle obtained in response to the change in the head.

4. The apparatus of claim 3, wherein the diagnostic unit determines that there is an abnormality in a vestibular function when the relative ratio calculated by the arithmetic unit is not “1.”

5. The apparatus of claim 1, further comprising a graph output unit configured to display the nystagmus detected by the nystagmus detection unit in three axes (horizon/vertical/torsion axes).

6. The apparatus of claim 1, further comprising a gaze guidance gazing point providing unit configured to provide a gazing point for gaze guidance through a display installed in the virtual reality device.

7. The apparatus of claim 6, wherein the gazing point has a curtain shape or a point shape.

8. The apparatus of claim 1, further comprising a posture adjustment guide unit configured to provide the patient with an accurate posture required for each test through the virtual reality device.

9. A method of diagnosing dizziness through eye movement measurement based on virtual reality, the method comprising:
(a) receiving a signal detected from a head tracker attached to a virtual reality device and detecting a head position and movement of a patient to detect the head movement of the patient;
(b) receiving raw data obtained by capturing the eye movement of the patient from an eye tracker attached to the virtual reality device to acquire and transmit eye movement video;
(c) distinguishing saccadic oscillation and nystagmus in the eye movement video and filtering meaningless eye movement that is not measured since an eye moves too slow or is covered due to blinking in the detected nystagmus to detect only meaningful nystagmus; and
(d) diagnosing a type of dizziness of the patient based on the detected meaningful nystagmus.

10. The method of claim 9, wherein, in the operation (b), the eye movement video is acquired and transmitted by processing the raw data of the eye movement captured using an internal camera of the eye tracker using a processing program.

11. The method of claim 9, wherein, in the operation (d), a relative ratio between the head movement and the eye movement is calculated by dividing a movement angle of a head detected in response to a change in a head in the operation (a) by the eye movement angle moving in response to the change in the head, and it is determined that there is abnormality in a vestibular function when the calculated relative ratio does not reach “1” or a normal value that is predetermined.

12. The method of claim 9, further comprising (e) displaying the nystagmus detected in the operation (c) in three axes (horizon/vertical/torsion axes).

13. The method of claim 9, further comprising, before the operation (b), (f) providing a gazing point for gaze guidance through the virtual reality device.

14. The method of claim 13, wherein the gazing point has a curtain shape or a point shape.

15. The method of claim 9, further comprising, after the operation (a), (g) providing the patient with an accurate posture required for each test through the virtual reality device.

16. A computer-readable recording medium in which a program for realizing the method of diagnosing dizziness through eye movement measurement based on virtual reality of claim 9 is stored.

17. A computer program stored in a computer-readable recording medium for realizing the method of diagnosing dizziness through eye movement measurement based on virtual reality of claim 9.
